(19) United States
(12) Patent Application Publication
(10) Pub. No.: US 2009/0069181 A1
(43) Pub. Date: Mar. 12, 2009

(54) Product and Process for Treating Water Bodies, Sediments and Soils

(75) Inventor: Noel BOULOS, Houston, TX (US)

Correspondence Address: Solvay;c/o B. Ortego - IAM-NAFTA, 3333 Richmond Avenue, Houston, TX, 77098-3099, US

(73) Assignee: SOLVAY SA, Brussels (BE)

(21) Appl. No.: 12/271,399

(22) Filed: Nov. 14, 2008

Related U.S. Application Data
(63) Continuation of application No. PCT/EP2007/055204, filed on May 29, 2007.
(60) Provisional application No. 60/809,022, filed on May 30, 2006.
(60) Provisional application No. 60/866,450, filed on Nov. 20, 2006.

(51) Int. Cl. C02F 1/72; A62D 3/33; A01N 59/00; C02F 5/08; C02F 3/12; C12N 1/20; A62D 3/38

(52) U.S. Cl. 504/151; 588/315; 588/320; 210/759; 435/253.6; 252/175; 210/620; 210/632

(57) ABSTRACT

A solid form of an inorganic peroxide product and associated process for the treatment, remediation, etc. of natural, man-made, industrial, municipal, etc., water bodies such as ponds, streams, lakes, canals, paddies, tanks, lagoons, pools, pipelines, etc, especially those that are contaminated, as well as in situ and ex situ treatment of sediment and soil.

### CROSS-REFERENCE TO RELATED APPLICATIONS

This application is a continuation of International Application No. PCT/EP2007/055204, filed on May 29, 2007, which claims priority to U.S. provisional application No. 60/809,022, filed on May 30, 2006, and U.S. provisional application No. 60/866,450, filed on Nov. 6, 2006, these application being incorporated herein in their entirety by reference.

### FIELD OF THE INVENTION

The present invention relates to an improved product and process for the treatment, remediation, etc. of natural, man-made, industrial, etc., water bodies such as ponds, streams, lakes, canals, paddies, tanks, lagoons, pools, pipelines, etc, especially those that are contaminated, as well as contaminated sediments, soils, sludges, manure, sewage, etc.

### BACKGROUND OF THE INVENTION

The use of calcium peroxide in the treatment of sediments is known in the art. See, e.g., “Bioremediation of sediments from intensive aquaculture shrimp farms by using calcium peroxide as slow oxygen release agent,” Environ. Technol. 2005 May; 26(5):581-9. However, in the past, a powder material has been used, which is difficult to handle and floats in water. The powder thus makes it difficult or impossible to handle when it is desired to treat large water bodies or the sediment or bottom, etc., of a water body when water is present without special effort and care, such as by draining the water.

The use of calcium peroxide in the ex situ treatment of soil contaminated with toxic spills is also well known in the art. However, calcium peroxide in a powder form is difficult to mix with the soil using state of the art machines. Generally, the powder product has ˜3-50 micron mean particle size.

### OBJECTS OF THE INVENTION

It is an object of the present invention to provide an improved product and process for the treatment, remediation, etc., of natural, man-made, industrial, municipal, etc., water bodies such as ponds, streams, drinking water reservoirs, wastewater, lakes, canals, paddies, tanks, lagoons, pools, pipelines, drainage ditches, aquaculture and aquafarming water bodies, rivers, seawater and freshwater aquaria, centers for breeding fish and other marine animals, manure storage lagoons from poultry, swine, cattle etc., water from sewage treatment plants and other water treatment plants etc. and any wastewaters including industrial wastewaters, the resultant waters also being able to comprise manure, sewage, soils, sediments and/or silts and sink and float matter etc, especially those that are contaminated and/or stagnant, such as and including sediments, soils, silts, sludge, solid waste, grease traps, etc.

### SUMMARY OF THE INVENTION

The present invention is directed to a solid form (e.g., granule, briquette, tablet, prill, flake etc.) and to the use of this product, where the product comprises at least one inorganic peroxide such as CaO₂, MgO₂ etc. and at least one corresponding inorganic hydroxide, oxide and/or carbonate. The term “corresponding” herein means containing the same metal, for instance the corresponding inorganic hydroxide of calcium peroxide is calcium hydroxide. This solid product is not a powder, and is engineered to have a bulk density such that it easily settles to the bottom of the water body to be treated and thus comes into contact with any residue, sediment, etc. located at the bottom of such water body. The product is easily mixed with soils or sediments for ex situ treatment. In addition, these product forms exhibit an oxygen release profile that is slower than the powder, leading to prolonged activity after a single application, thus reducing the frequency of re-application.

The present invention is further directed to a combination product for treating a natural, man-made, industrial, municipal, etc., water body such as a pond, a stream, a lake, a canal, a paddy, a tank, a lagoon, a pool, a pipeline, a drainage ditch, aquaculture or aquafarming water body, manure storage lagoons from poultry, swine, cattle, sewage etc, especially those that are contaminated and/or stagnant, comprising: a solid form such as a briquette, a tablet, granules, prills, flakes, pellets, etc., comprising at least one inorganic peroxide and at least one corresponding inorganic hydroxide, oxide and/or carbonate and optionally including one or more binders, extenders, adjuvants, buffers, stabilizers etc., and any one or more of: metals or compounds thereof, preferably one or more transition metals and/or compounds thereof, algaecides, enzymes, microbes, herbicides, zeolites, silicates, inorganic compounds such as calcium carbonate, sodium carbonate, bicarbonate or sesquicarbonate, magnesium salts, silicates, aluminosilicates, other peroxides, and mixtures thereof, said solid form preferably having a bulk density greater than 0.65 g/cm³, the ingredients of the combination product being present in admixture or separately in one or more containers.

The present invention is also directed to a process for treating a natural, man-made, industrial, municipal, etc., water body such as a pond, a stream, a lake, a canal, a paddy, a tank, a lagoon, a pool, a pipeline, a drainage ditch, aquaculture or aquafarming water body, manure storage lagoons from poultry, swine, cattle etc, especially those that are contaminated and/or stagnant, as well as for treating contaminated sediments and soils, etc. at the bottom of a water body, and sewage, comprising: adding to said water body, sediment, soil, etc. at least one solid form such as a briquette, a tablet, granules, prills, flakes, pellets, etc., comprising at least one inorganic peroxide and at least one corresponding inorganic hydroxide, oxide and/or carbonate optionally including one or more binders, extenders, adjuvants, buffers, stabilizers etc., said solid form having a bulk density such that the solid form sinks in the water body, optionally in conjunction with any one or more of: metals or compounds thereof, preferably one or more transition metals and/or compounds thereof, algaecides, enzymes, microbes, herbicides, zeolites, silicates, inorganic compounds such as calcium carbonate, sodium carbonate, bicarbonate or sesquicarbonate, magnesium salts, silicates, aluminosilicates, other peroxides, and mixtures thereof.

The present invention is also directed to a process for ex situ treatment of contaminated sediments and soils comprising: adding at least one solid form such as a briquette, a tablet, granules, prills, flakes, pellets, etc., the solid form comprising at least one inorganic peroxide and at least one corresponding hydroxide, oxide and/or carbonate and optionally including one or more binders, extenders, adjuvants, buffers, stabilizers etc., with a particle size at least 100 microns in average diameter.

### BRIEF DESCRIPTION OF THE DRAWINGS

For a detailed description of the invention, reference will now be made to the accompanying drawings in which:

FIG. 1 is an image of a pond being laden with blue green algae before treatment;

FIG. 2 is an image of the same pond as in FIG. 1 after application of a product according to one embodiment of the present invention;

FIG. 3 is an image of another pond being laden with blue green algae and smells of hydrogen sulfide before treatment;

FIG. 4 is an image of the same pond as in FIG. 3 after application of a product according to another embodiment of the present invention;

FIG. 5 is an image of a water feature shown before treatment with an algal bloom after an aeration pump failure;

FIG. 6 is an image of the same water feature as in FIG. 5 two weeks after application of a product according to yet another embodiment of the present invention; and

FIG. 7 is an image of the same water feature as in FIG. 5 four weeks after application of a product according to the present invention.

### DETAILED DESCRIPTION OF THE PREFERRED EMBODIMENTS

In the present invention, an inorganic peroxide such as CaO₂ (calcium dioxide, calcium peroxide, calcium peroxygen), strontium peroxide, barium peroxide, zinc peroxide, cadmium peroxide, sodium peroxide, and magnesium peroxide, etc., including mixtures thereof, is prepared in solid form such as in the form of briquettes, tablets, granules, flakes, prills, etc., optionally with binders, extenders, adjuvants, buffers, stabilizers, etc., in such a manner that the solid form preferably sinks in water. In this regard, the bulk density of the solid form is important. The term “solid form” as used herein does not include powder form. Calcium peroxide is the preferred inorganic peroxide.

Bulk density is calculated by dividing the solid form's mass by its volume. Thus, bulk density of the solid form can be altered by changing the solid form's mass (adding to it or taking away from it) and/or by changing its volume (increasing or decreasing the amount of space taken up by the form).

In the present invention the solid form chosen is not limited with regard to shape, size, components, etc., as long as it contains at least one inorganic peroxide. Granules are preferred, as are briquettes, flakes, prills and tablets. As noted above, the solid forms herein can include extenders (talc, etc.), adjuvants, buffers (bicarbonate, etc.), stabilizers, and binders (silicate, etc.).

Especially for granules and flakes but also for the other forms, a typical preferred size of the solid form is, e.g., 0.01, 0.05, 0.08, 0.1, 0.2, 0.4, 0.5, 0.7, 0.9, 1.0, 1.3, 1.5, 2, etc. centimeters in average size such as diameter. The average size is generally higher than or equal to 0.005 cm, in particular higher than or equal to 0.006 cm, in most cases higher than or equal to 0.007 cm. The average size is commonly lower than or equal to 5 cm, especially lower than or equal to 4 cm, values lower than or equal to 3 cm being common. Average sizes from 0.01 to 2 cm give good results. The average size (or D₅₀) is determined by measuring the size distribution from Laser Scattering Particle Size Distribution Analysis, D₅₀ being the particle size value which expresses that 50% by mass of the particles have a size value lower than or equal to D₅₀. Powders are excluded.

The solid forms are generally agglomerates, aggregates, clusters or any other form made up of primary particles. Primary particles are defined as the smallest discrete particles that can be seen by Electron Microscopy analysis. These primary particles have generally a mean particle size higher than or equal to 1 micron, in particular higher than or equal to 3 micron, values higher than or equal to 10 micron being common. The mean particle size of the primary particles is usually lower than or equal to 500 micron, especially lower than or equal to 400 micron, values lower than or equal to 300 micron being possible. The mean particle size of the primary particles is preferably from 3 to 20 microns. Particularly preferred sizes of the primary particles useful herein are greater than 3 micron mean particle size, for example greater than 5, 10, 15, 20, 30, 50, 100, 200, 300 etc. micron mean particle size. The mean primary particle size is measured indirectly through the measurement of the specific surface area.

The bulk density of the solid forms can be altered as noted above by changing the mass of the solid form (e.g., by altering the ingredients thereof) and/or changing its shape and/or its degree of compaction (e.g., more or less compact, etc.), or the choice of process to generate the solid form (e.g., extrusion, agglomeration, compaction, pelletization etc.). Bulk densities of the solid forms herein are preferably greater than 0.65 g/cm³, in particular greater than or equal to 0.7 g/cm³, for instance greater than or equal to 0.8 g/cm³, especially greater than or equal to 0.9 g/cm³, usually greater than or equal to 1.0/cm³, in some cases greater than or equal to 1.1 g/cm³, up to the physical density of the product. The bulk density of the solid form is usually lower than or equal to 1.5 g/cm³, in particular lower than or equal to 1.4 g/cm³, in special cases lower than or equal to 1.3 g/cm³, for instance lower than or equal to 1.2 g/cm³. In a highly preferred embodiment, the bulk density of the solid form is from 0.8 to 1.2 g/cm³. The bulk density is measured according to the standard DIN53194.

Preparation of the solid forms herein can be effected by art accepted methods, such as tableting, compaction, pelletization, agglomeration, prilling etc.

Preferably, the invention solid forms sink in the water body being treated (sinking solid forms). Preferably all of the solid form used sinks, but it is acceptable that less than 100% sinks. It is preferred that more than 50% sinks, including more than 60, 70, 80, 90, 95 and 99%. The sinking rate is not limited, but is preferably relatively fast so as to insure good control of the placement of the solid form. In this regard the solid form generally sinks at a rate of at least 0.001 meter per second, for instance at least 0.005 m/s, in particular at least 0.05/s, especially at least 0.1 m/s, in many cases at least 0.2 m/s, in special cases at least 0.3 m/s, possibly at least 0.5 m/s, commonly at least 0.7 m/s, often at least 0.9, preferably at least 1 m/s, and most preferably at least 2 m/s. The measurement of the sinking rate can be conducted for example in a clear column of room temperature water having a diameter that is at least 12 times the size of the solid form so as to minimize any edge/side effects of the column on the sinking rate. A sinking rate of about 1 ft/5 seconds or 0.06 m/second gives good results. The solid form sinks in water at temperatures of from 0° C. to 99° C.

The solid form used in the invention generally contains the inorganic peroxide in an amount which is usually higher than 25% by weight, in particular equal to or higher than 30% by weight, for instance higher than 35% by weight, often equal to or higher than 40% by weight, advantageously equal to or higher than 50% by weight, especially equal to or higher than 60% by weight, in many cases equal to or higher than 65% by weight, commonly equal to or higher than 70% by weight, for instance equal to or higher than 75% by weight, the remainder being substantially (except for possible impurities) the corresponding metal oxide or hydroxide and/or the corresponding metal carbonate and optionally adjuvants, fillers, binders, buffers, stabilizers, etc. The amount of metal peroxide in the particles is most often equal to or smaller than 80% by weight, especially equal to or smaller than 70% by weight. The remainder is usually the corresponding metal oxide or hydroxide and/or the corresponding metal carbonate and optionally other adjuvants, fillers, binders, buffers, stabilizers, etc. A greater concentration of the inorganic peroxide in the solid form leads to better performance or to the same performance at a lower dose by weight. Using solid forms having more than 25% by weight of inorganic peroxide, especially calcium peroxide, requires a lower dose than the more dilute forms. This is an advantage in the practical use of the solid form leading to a lower amount of chemical addition to the water body.

The inorganic peroxide, before and/or after being transformed into the solid form, can be coated or uncoated. A coating can be advantageous when the inorganic peroxide as such dissolves too fast in water to perform the desired effect. Uncoated inorganic peroxide, and especially uncoated calcium peroxide, is preferred. It has indeed been found that reducing the surface area of the solid form by granulation reduces its dissolution rate to an acceptable level, and coating of the solid form of the current invention is typically not needed. This is in contrast to the powder which would require coating in order to achieve a similar reduction in its dissolution rate. The solid form used in the invention can be coated if it is desired to further suppress its dissolution rate or the primary particles of which the solid form is made could be coated. The inorganic peroxide can also be supported on a carrier or be unsupported. Unsupported inorganic peroxide, and especially unsupported calcium peroxide, is preferred.

The solid form used in the invention can contain additives. However, it is preferred that these additives are not organic materials (because they can be hazardous in the presence of inorganic peroxides), transition metal compounds (because they destabilize peroxides) and phosphates (because adding phosphate defeats the purpose of immobilizing the phosphate already present in the water body). Additives can be any compound compatible with the inorganic peroxide and the application in which the solid form will be used, including calcium derivatives such as calcium hydroxide, oxide, or carbonate, silicates such as sodium silicate and metasilicate, aluminosilicates such as clay, kaolin, bentonite and montmorillonite, buffers such as sodium bicarbonate, stabilizers such as silicates, other peroxides such as magnesium peroxide and sodium carbonate peroxyhydrate, magnesium salts, binders etc. A solid form consisting essentially of the inorganic peroxide and the corresponding metal oxide or hydroxide and/or the corresponding metal carbonate is preferred. In the case of calcium peroxide, the solid form preferably consists essentially of calcium peroxide and calcium hydroxide.

The solid form of this invention has a much lower surface area per unit weight than the primary particles. This contributes to a reduction in the dissolution rate of the active ingredients embedded in the solid form.

The solid form of this invention also allows the product to be dispensed over a body of water using spreaders. As the particle size increases, the range of reach of these spreaders also increases thus facilitating application over large bodies of water while achieving more even distribution of the product over the water and at the sediment or bottoms.

Preferred sinking solid forms include the following:
A sinking form such as a granule that does not disintegrate quickly or easily (e.g., due to a high degree of compaction or the presence of binders such as sodium bicarbonate, silicate, etc.)
A sinking form such as a granule with an oxygen release profile that is slower than the same material in powder form
A sinking form such as a granule comprising at least one inorganic peroxide that generates both oxygen and hydrogen peroxide
A sinking form such as a granule that is easy to spread over wide areas of bodies of water to facilitate its application and provide homogeneous dosing.
A sinking form such as a granule that optionally contains a buffer or is mixed with a granular buffering compound to avoid large pH fluctuations.

Preferred solid forms useful herein are described in U.S. Patent Publication US2005239679 and WO 2004 035 470, both incorporated herein by reference.

The amount of inorganic peroxide used herein is not limited and depends on the amount and type of water or soil or sediment, etc. to be treated, extent of contamination, the desired degree of remediation, type of benefit desired, etc. For example, if phosphate immobilization is a goal, then a phosphate-immobilization-effective amount of the one or more inorganic peroxides is added to the, e.g., water body to be treated for the control of aquatic vegetation and algae. This amount is easily determined by one of skill in this art based on this description, and is an amount that immobilizes some or all of the phosphate. For example, phosphate measurements can be taken before and after addition of the one or more inorganic peroxides to confirm some or complete immobilization. If remediation of a toxic organic contaminant is desired, then much higher dosing is usually required. This can be determined by measuring the amount of contaminant and calculating the amount of oxygen necessary to oxidize the contaminant based on chemical principles and practices used in the remediation and bioremediation of toxic organic compounds in the soil and groundwater remediation industry.

For a 70% inorganic peroxide concentration, the amount of solid form used is generally higher than or equal to 0.1 lb/acre, in particular higher than or equal to 0.5 lb/acre, especially higher than or equal to 1 lb/acre, values of at least 10 lb/acre giving good results. The amount of solid form is usually lower than or equal to 10,000 lb/acre, for instance lower than or equal to 5,000 lb/acre, in many cases lower than or equal to 1,000 lb/acre, values lower than or equal to 500 lb/acre being advantageous. Useful amounts of solid forms, sinking and otherwise, include, e.g., from 1 lb/acre to 1,000 lbs/acre of granular inorganic peroxide. For more dilute formulations, the amount is adjusted to provide the same amount of inorganic peroxide in the application.

For use in soils, sediments, sludge, sewage, etc., useful amounts include 1 ppm, 5 ppm, 10 ppm, 20 ppm, 50 ppm, 100 ppm, 1000 ppm, 10,000 ppm, etc. of medium being treated. Those of ordinary skill in this art can determine the amount of inorganic peroxide to use based on this disclosure. For more dilute formulations, the amount is adjusted to provide the same amount of inorganic peroxide in the application.

In ponds, streams, lakes, canals, paddies, tanks, lagoons, pools, pipelines, drainage ditches, etc, especially those that are stagnant and/or contaminated, anaerobic degradation of organic matter leads to water fouling and the production of H₂S, NH₃, methane, etc. In addition, Fe (III) phosphate complexes in the sediment are reduced to Fe (II), releasing phosphate into the water and increasing algae growth. Water, soil, sediment, sludge, etc. can also contain other toxic contaminants. Contaminants treated herein include various man-made and naturally occurring organic and inorganic products such as sulfides, nitrites, transition metals, organic matter naturally occurring in water, sediments, sewage and manure as well as organic compounds resulting from pollution, spills etc. such as chlorinated compounds, e.g., volatile organic compounds such as chlorinated olefins including tetrachloroethylene, trichloroethylene, cis 1,2-dichloroethane and vinyl chloride, and non chlorinated solvents such as methylethylbenzene, tertiary butyl alcohol, and methyl tert-butyl ether (MTBE). Other compounds include aromatic or polyaromatic ring compounds such as benzene, toluene, methylbenzene, xylenes, naphthalene, dichlorobenzene and propellents or explosives such as nitroanilines, trinitrotoluene, and so forth. The groups of compounds characterized by aromatic ring structures also include alkyl substituted aromatic hydrocarbons. Metals such as mercury, etc., pesticides, PCBs, and DDT are included.

These problems, among others, are treated effectively by the solid forms described herein, which when added to water bodies such as ponds, streams, lakes, canals, paddies, tanks, lagoons, pools, pipelines, drainage ditches, aquaculture and aquafarming water bodies, manure storage lagoons from poultry, swine, cattle, sewage etc, especially those that are contaminated and/or stagnant, provide the following benefits:
Easy to handle product which can be spread over large areas with minimum effort, reduces dust formation during application, quickly settles in the sediment, and is difficult to re-suspend in water due to water currents etc.
Granules that settle to the bottom of the water body to be treated and provide a point source of active ingredients in contact with or in proximity to any sediment, bottoms etc. of the water body being treated for bigger impact where these chemicals are needed
Extended release active ingredients at the sediment, bottoms, etc. due to the solid form's size and reduced surface area, compared to the powder
Slow release of oxygen (replenishing dissolved oxygen in order to sustain living organisms in water such as fish; oxidation of sulfides, nitrites, etc. in sediment and water; conversion of Fe(II) to Fe(III) complexes in the sediment leading to phosphate immobilization reducing nutrients in water; increased aerobic microbial population leading to higher aerobic biodegradation of organic matter and toxic contaminants and enhanced conversion of ammonia to nitrite)
Slow release of hydrogen peroxide (oxidation of sulfides, nitrites, etc. in sediment and water; oxidation of color bodies; degradation of chlorine in water thus preventing the formation of toxic chlorinated organic compounds; oxidation of toxic contaminants either directly or through the generation of hydroxyl free radicals by interaction with Fe ions; oxidation of Fe(II) to Fe(III) which forms phosphate complexes leading to phosphate immobilization; oxidation of pyrite which releases more Fe(III) in water which in turn immobilizes phosphate)
Slow release of alkalinity (reaction with anions such as free hydrogen sulfide, acidity in acid rain, sulfuric acid produced from sulfide oxidation, carbon dioxide, and organic acids obtained from degradation of organic matter)
Slow release of corresponding metal ions in water e.g. Ca from calcium peroxide (reaction with anions such as nitrate, carbonate, carbon dioxide, sulfate, sulfide, phosphate etc. leading to insoluble salts which reduces acidity, leads to phosphate immobilization, reduces photosynthesis due to reduction in CO₂, and can lead to reduced water conductivity; providing a source of calcium that is absorbed by living organisms to sustain life)

Some preferred uses and benefits of the invention include:
Maintenance of a good level of dissolved oxygen in water bodies especially at sediment, despite high summer temperatures that reduce oxygen solubility in water, and very low winter temperatures that lead to ice covering on water bodies preventing oxygen absorption from atmosphere.
High overall water quality that encourages a diversity of plant and animal life, enhances the recreational value of the body of water, and prevents fish kills.
Healthier fish population which thrives in better quality water, especially bottom dwelling creatures such as catfish and shrimp with increase in feeding rate leading to larger fish.
Reduced acidity by reaction with acids such as organic acids especially in aquaculture ponds improves water quality for fish
Reduction in the amount organic matter such as animal waste and sewage
Enhanced aerobic microbial degradation of organic matter leading to a reduction in BOD (Biological Oxygen Demand) in water, sediment, manure, sewage etc.
Enhanced growth of nitrifying bacteria which convert ammonia to nitrite, leading to reduced ammonia odor.
Enhanced oxidation of organic matter such as mercaptans leading to reduction in Chemical Oxygen Demand (COD) and toxicity.
Enhanced water clarity.
Reduction in anaerobic bacteria thus preventing anaerobic degradation of organic matter and avoiding putrification.
Reduction of filamentous bacteria thus causing debulking.
Enhanced zooplankton population due to higher assimilation of products obtained from aerobic degradation.
Immobilization of heavy metals such as Fe, Mn, Cu and As.
Oxidation of inorganic matter such as nitrites and sulfides
Odor control such as control of H₂S and NH₃, including elimination of unpleasant odors (oxidation of reduced sulfur species and elimination of ammonia produced under anaerobic conditions)
Better pH control (alkalinity prevents accumulation of organic acids and carbon dioxide and reacts with other anions in water)
Long term reduction of eutrophication through phytoplankton and algae control without the need for frequent re-application. While not bound by theory, the mechanism of action is through the slow release of oxygen and hydrogen peroxide at the sediment level which maintains Fe and Mn in an oxidized state. The oxidized form of these metals complexes with the phosphate in water and is insoluble, so P is immobilized in the sediment. Phosphate immobilization deprives algae from nutrients. The slow release of Ca from calcium peroxide may also contribute to form insoluble Ca phosphate complexes, further immobilizing phosphate. Due to the extended effect on algae control, this approach is not only very economical, but also avoids the addition of toxic algaecides to water.
Reduction of off flavor in catfish in aquaculture ponds. The elimination of algae prevents the release of chemicals from algae that are absorbed by catfish and give them an off flavor preventing their harvest. This shortens the cycle of catfish production and enhances profitability of aquaculture farms.
Sediment is maintained in an oxidized state just below the surface throughout all the seasons, which prevents dangerously low dissolved oxygen in bottom water when sediment is disturbed.
Reduced toxicity of water, sediment, sludge, etc. due to elimination of many of the toxic chemicals through either biodegradation or direct oxidation of these chemicals, or because maintaining an aerobic state prevents these chemicals from forming.
Reduced sediment levels due to enhanced biodegradation of organic matter can eliminate the need for expensive dredging. Dredging is undesirable not only because it is expensive, but also because it releases toxic metals in water during the dredging operation. If dredging is still necessary, the sediment dredged would be of reduced toxicity and could be reused e.g., as a fertilizer or for another application.
Reduction of sediment and water toxicity and optionally destruction of toxic compounds such as pesticides and spills of organic contaminants
Sludge, sewage, soil, water, etc. can be more easily or more quickly disposed of due to its reduced toxicity and aerobic conditions, or reused.
In situ remediation of contaminated sediments and ex situ remediation of sediments, soils, sludge, etc. from contamination from chemical spills such as diesel, pesticides etc spills leading to reduced toxicity of water, sediment, sludge, etc. Preferably, a Fenton-like process (M+H₂O₂=>HO.+HO⁻+Mⁿ⁺+(n−1)e⁻) is used to generate hydroxyl free radicals wherein M is Fe or another metal and n is any value from 1 to 7. Methods of hydroxyl free radical generation using hydrogen peroxide described in C. Walling, Acc. Chem. Res., 8 (1975) 125 and U.S. Pat. No. 6,692,632 are incorporated herein by reference.

Two or more of these uses can of course be combined.

The primary particles used to make the solid form of this invention can include inorganic peroxides such as calcium peroxide, for instance the commercial product IXPER® 60C or 75C of SOLVAY SA (approximately 60-75% CaO₂, the balance being primarily calcium hydroxide and calcium carbonate, having a bulk density of 0.4-0.65 g/cm³, and a mean primary particle size of 3-50 microns), magnesium peroxide, for instance the commercial product IXPER® 35M of SOLVAY SA (approximately 35% magnesium peroxide, the balance being primarily magnesium oxide, having a bulk density of 0.65-0.75 g/cm³, and a mean primary particle size of 3-50 microns), strontium peroxide, barium peroxide, zinc peroxide, cadmium peroxide, sodium peroxide, etc

The peroxides and supplemental actives described in U.S. Pat. No. 6,569,342, incorporated herein by reference, can be used herein. A preferred solid form comprises, e.g., a combination of CaO₂, Ca hydroxide and CaCO₃ optionally with a stabilizer such as silicates, and a binder such as NaHCO₃. Another preferred combination of agents, used together in the same solid form or together in a process but not in the same solid form added before, concurrently or after the addition of the solid form include: at least one inorganic peroxide combined with one or more of another inorganic peroxide such as sodium percarbonate (PCS), Na₂CO₃, CaCO₃, Ca(OH)₂, silicates such as sodium silicate, silicone dioxide, aluminosilicates such as clay, magnesium products such as magnesium oxide, living organisms such as aerobic bacteria alone or supported on an inert ingredient, optionally coated, or enzymes.

In addition, other reactants such as those used in a hydroxyl free radical generation reaction (e.g., Fenton reaction; see, e.g., C. Walling, Acc. Chem. Res., 8 (1975) 125, incorporated herein by reference, for a description of the Fenton Process) can be dispersed with the inorganic peroxide-containing solid form separately either before, during, and/or after dispersion of the inorganic peroxide (e.g., a metal chelate and/or a metal compound such as a metal oxide or a salt of an inorganic or organic acid such as iron citrate).

Again without being bound by theory, it is believed that the role of inorganic peroxides such as calcium peroxide in control of eutrophication is to remove or reduce the amount of phosphorus present in any form (e.g., phosphates, etc.), in water bodies by maintaining metals such as Fe and Mn in an oxidized state. It is the metals that immobilize P. If there is not enough metal(s) in the sediment, then it is advantageous, but counterintuitive, to add more. For example, FeCl₃ is used with advantage for P immobilization, where its role would be oxidation and also conversion of PO₄ to FePO₄. The addition of a material or materials that can precipitate P, such as PO₄, before, with, and/or after the application of inorganic peroxide(s) such as calcium peroxide provides several advantages, such as the removal of the PO₄ that is soluble in water faster. Other compounds that can be added to this purpose include other iron derivatives and other transition metal compounds such as manganese etc.

These materials are preferably added as oxides, hydroxides or salts, especially their halogen, etc., salts. Whether or not such material(s) is/are added, it may be preferable to adjust pH to a preferred range of 2-12, including 2.5, 3, 4, 5, 6, 7, 8, 9, 10 and 11 and all values and subranges between all stated values. For example, once FeCl₃ reacts with PO₄ it leaves behind HCl.

In another preferred embodiment of the invention, one or more inorganic peroxides can be used in conjunction with one or more algaecides. This addition can be before and/or after and/or during addition of the one or more inorganic peroxides. Examples of algaecides are sodium percarbonate, copper sulfate, and chelated Cu.

For example, one protocol could be:
First add an algaecide selected from, e.g., sodium carbonate peroxyhydrate, CuSO₄ . . . to treat algae. This preferably gets rid of some or all of the algae immediately
Add, e.g., CaO₂ to immobilize PO₄ and prevent more algae from forming.

In another preferred embodiment of the invention, one or more inorganic peroxides can be used in conjunction with one or more microbes. This addition can be before and/or after and/or during addition of the one or more inorganic peroxides.

In another preferred embodiment of the invention, one or more inorganic peroxides can be used in conjunction with one or more aquatic herbicides. This addition can be before and/or after and/or during addition of the one or more inorganic peroxides Useful aquatic herbicides include fluridone (Sonar), 2,4D (2,4-dichlorophenoxyacetic acid), or a slimicide.

In another preferred embodiment of the invention, one or more inorganic peroxides can be used in conjunction with one or more silicate or aluminosilicate such as zeolites and clays. This addition can be before and/or after and/or during addition of the one or more inorganic peroxides. This is particularly effective when special zeolites are used which lead to ammonia removal.

In another preferred embodiment of the invention, one or more inorganic peroxides can be used in conjunction with one or more of another inorganic peroxide oxidizer to boost performance. Examples include metal percarbonates, H₂O₂, etc. CaO₂ is sometimes referred to as alkaline earth metal peroxide to differentiate it from other inorganic peroxides.

The solid form product may be used with another method to increase dissolved oxygen such as mechanical aeration, or addition of a peroxide such as sodium percarbonate or hydrogen peroxide.

The solid form product may be used with a second oxidizer to effect the oxidation of toxic organic contaminants, the second oxidizer being chosen from sodium percarbonate, hydrogen peroxide, sodium persulfate, potassium persulfate, and their mixtures, this oxidizer being added before, with or after the solid form product, optionally by using in addition a product to adjust the pH, such as sodium carbonate or sodium hydroxide. A metal compound such as a salt can be added before, with or after the other products, preferably Fe compounds such as Fe EDTA, Fe oxide, or FeCl₃.

In another preferred embodiment of the invention, one or more inorganic peroxides can be used in conjunction with one or more of Ca or Mg hydroxide, carbonate or oxide, or sodium carbonate, sesquicarbonate or bicarbonate etc.

Of course, all of the above additional additives (algaecide, herbicide, microbe, etc.) can be used in combination all together or in any selected possible combination. Also, sequential treatments of the same or different combinations of agents (or inorganic peroxide alone) may be used with, e.g., aeration of the water body being treated (e.g., aeration during, before, or after treatments with different actives, etc.).

The combinations described may be in one solid form (i.e., a granule containing an inorganic oxide and another component as listed above), or they may make a simple mixture, or they may be a solid form of inorganic oxide (e.g., granule) mixed with another component, or they may be combined in a kit and not physically mixed.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

### EXAMPLES

### Example 1

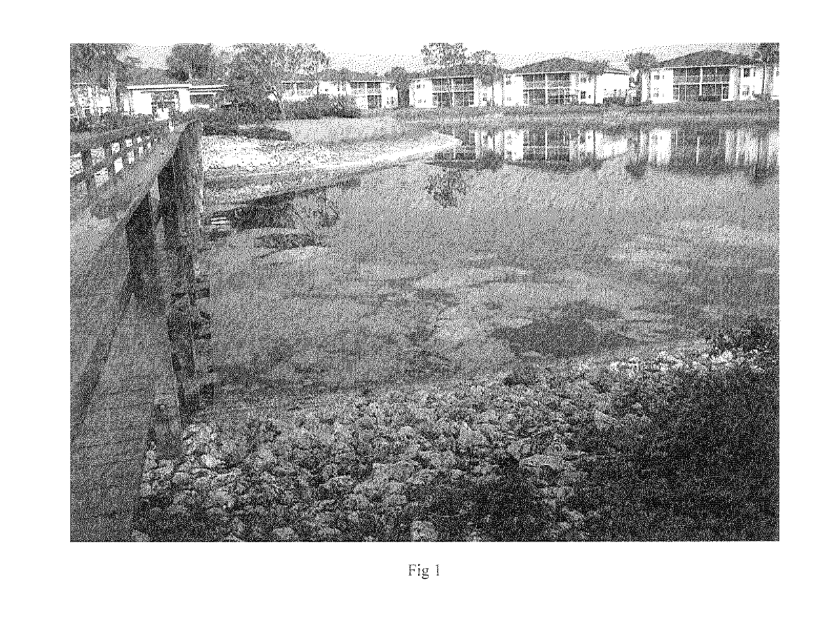

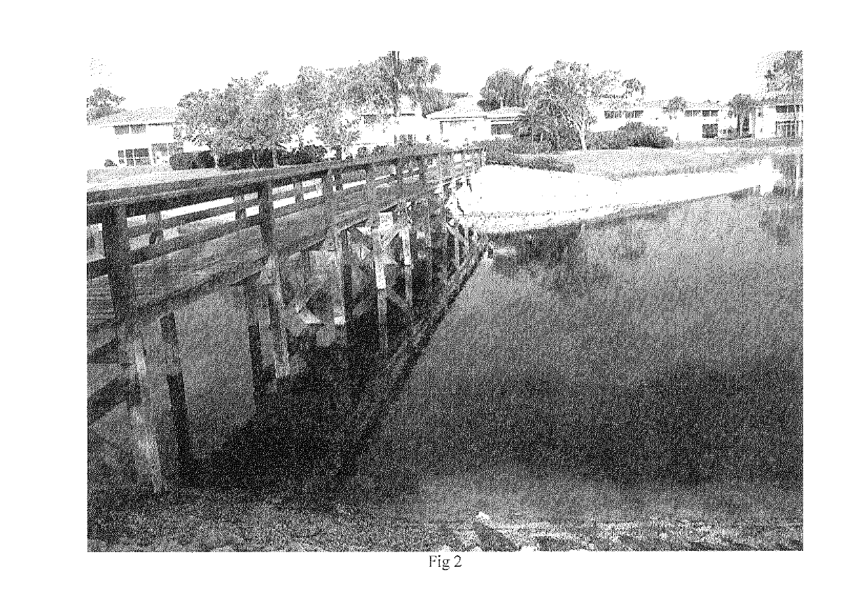

A four-foot deep pond had a seven year history of being laden with blue green algae. It had been treated every two weeks with copper sulfate algaecide for approximately 6 years during the warmer season, but control of algae was still difficult. At the beginning of the summer of 2006, the dissolved oxygen (DO) at the bottom of this pond was 2 ppm. The solid form of this invention containing approximately 70% calcium peroxide was applied to the pond by spreading it over the surface at a rate of 10 lbs per acre foot. The following day, DO at the bottom had gone up to 8.6 ppm. Pictures were taken of the pond just before application showing blue-green algae growth and turbidity (see FIG. 1). Another picture (see FIG. 2) was taken 4 weeks later which showed the pond was clear and free from algae. The pond remained without algae throughout the summer without the need to add another dose of the solid form of the invention or any other product.

### Example 2

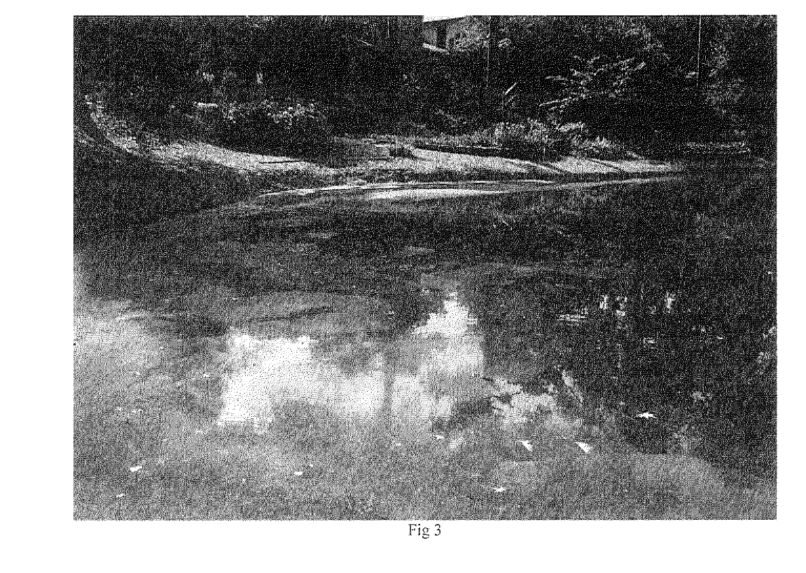

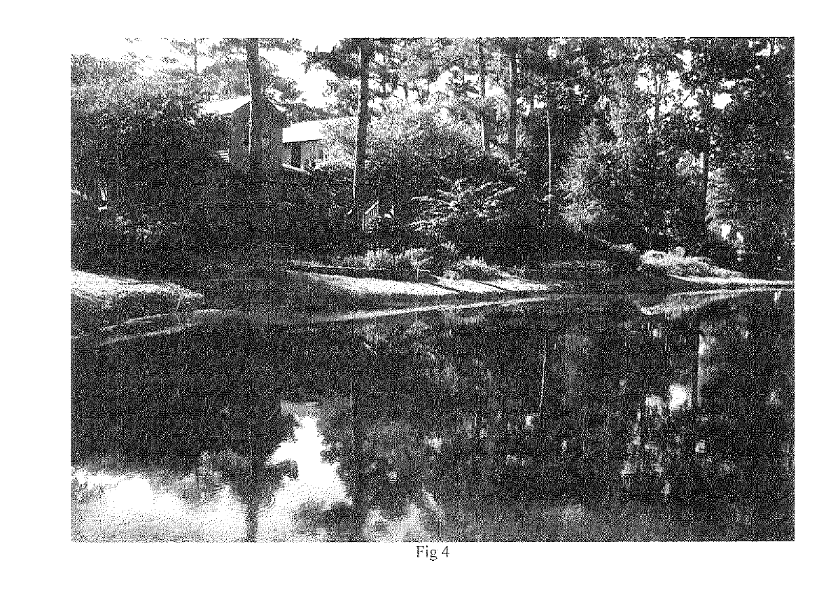

A pond, approximately three-foot deep, had a history of being laden with algae and smells of hydrogen sulfide for many years. The solid form of this invention containing approximately 70% calcium peroxide was applied at a rate of 40 lbs per surface acre by spreading over the surface. Pictures were taken before application (see FIG. 3) and after (see FIG. 4) a two-week period. Observations were that the algae from both the pond surface and the water column were eliminated. The clarity improved dramatically during this two-week period.

### Example 3

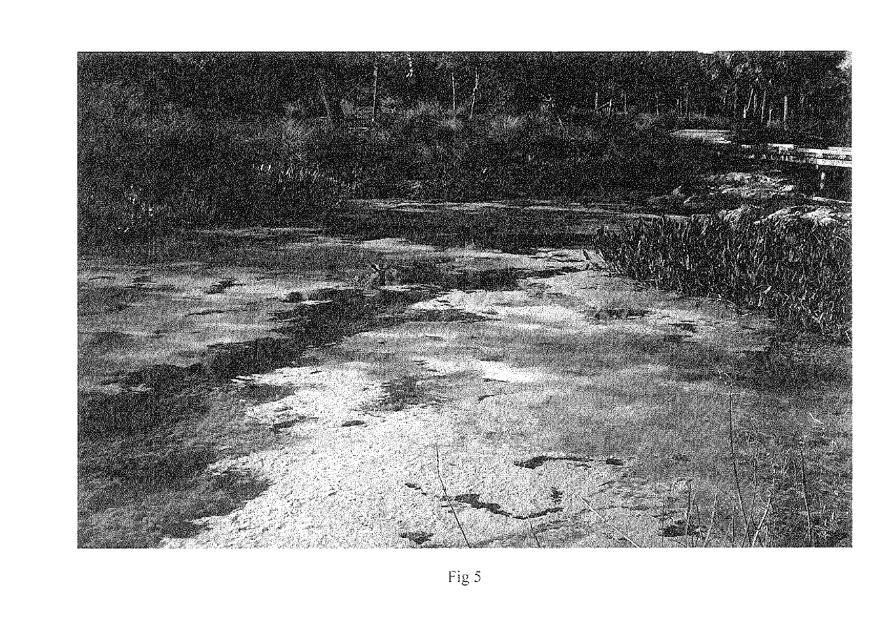

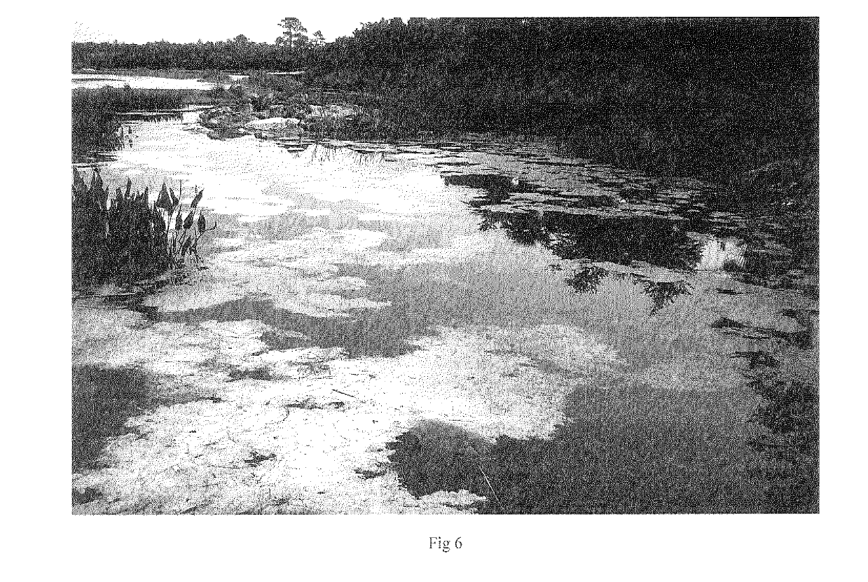

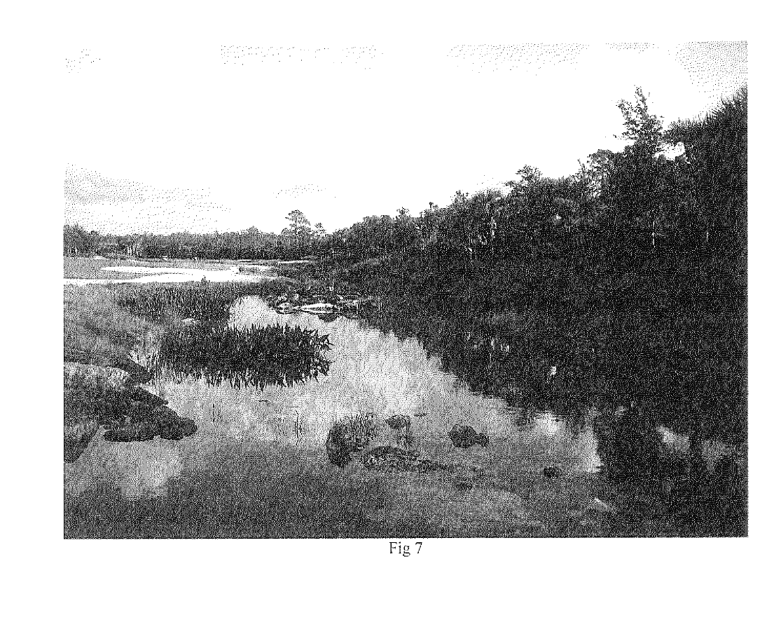

This pond was a water feature that had a fountain for aeration. After the pumps failed, the nutrient inload quickly overloaded the area and algae began to bloom. This led to an anaerobic state as well as high BOD. It had been treated with Copper Sulfate several times and had a high amount of organic matter on the bottom. Treatment involved spreading the solid form of this invention containing approximately 70% calcium peroxide at a rate of 60 lbs per surface acre. Pictures were taken before treatment (see FIG. 5) and two weeks later (see FIG. 6). It was noticed that the algae were dead after the first two weeks, however because the mats were so thick they did not sink. After 4 weeks from application (see FIG. 7), more pictures were taken and all of the algae had dissipated and the area did not need to be treated the rest of the summer.

1. A process for treating a water body or treating a sediment or soil at the bottom of a water body, comprising:
adding to said water body, sediment, or soil at least one solid form comprising at least one inorganic peroxide and at least one corresponding inorganic hydroxide, oxide and/or carbonate, said solid form optionally including one or more binders, extenders, adjuvants, buffers, or stabilizers, said solid form having a bulk density such that the solid form sinks in the water body, optionally in conjunction with any one or more additives selected from the group consisting of: metals or compounds thereof, algaecides, enzymes, microbes, herbicides, zeolites, silicates, inorganic compounds, other peroxides, and mixtures thereof.

2. The process according to claim 1 wherein the treatment is for phosphate immobilization.

3. The process according to claim 1 wherein the treatment is for control of aquatic vegetation and algae including their destruction or prevention of their growth.

4. The process according to claim 1 wherein the treatment is for increasing aerobic bacteria, enhancing aerobic biodegradation of organic matter, and reducing BOD.

5. The process according to claim 1 wherein the treatment is for odor control.

6. The process according to claim 1 wherein the treatment is for an improvement to the living environment of fish, shrimp, and their eggs leading to better feeding rates and bigger fish.

7. The process according to claim 1 wherein the treatment is for heavy metals immobilization.

8. The process according to anyone of claim 1 wherein the treatment is for reduction of CO₂ in water.

9. The process according to claim 1 wherein the treatment is for pH control in order to counter the effect of acid rain and organic acids produced from the degradation of organic matter.

10. The process according to claim 1 wherein the treatment is for reduction of the amount of sediment to reduce the need for dredging.

11. The process according to claim 1 wherein the treatment is for reduction in the amount of organic matter.

12. The process according to claim 1 wherein the treatment is for enhancing water clarity, and improving water color.

13. The process according to claim 1 wherein the treatment is for reduction of sediment and water toxicity and optionally for the destruction of toxic compounds and spills of organic contaminants.

14. The process according to claim 1 wherein the treatment is for oxidation of nitrites or sulfides.

15. The process according to claim 1 wherein the treatment is for oxidation of organic matter, and reduction of COD.

16. The process according to claim 1 wherein the treatment is for reducing anaerobic bacteria.

17. The process according to claim 1 wherein the treatment is for reducing water conductivity.

18. The process according to claim 1 wherein the treatment is for reducing catfish off-flavor in aquaculture farms.

19. The process according to claim 1 wherein the treatment is for enhanced dissolved oxygen in the sediment which reduces sediment volume and toxicity, and reduces deleterious effect on water quality during dredging.

20. The process according to claim 1 wherein the solid form is granules having a mean particle size of at least 100 microns.

21. The process according to claim 1 wherein the inorganic peroxide is at least one peroxide selected from the group consisting of calcium peroxide, strontium peroxide, barium peroxide, zinc peroxide, cadmium peroxide, sodium peroxide, and magnesium peroxide.

22. The process according to claim 21 wherein the inorganic peroxide is a peroxide selected from the group consisting of calcium peroxide, magnesium peroxide, zinc peroxide, sodium peroxide, and mixtures thereof.

23. A combination product for treating a water body, comprising:
a solid form comprising at least one inorganic peroxide and at least one corresponding inorganic hydroxide, oxide and/or carbonate, and optionally including one or more binders, extenders, adjuvants, buffers, stabilizers, and any one or more additives selected from the group consisting of: metals or compounds thereof, algaecides, enzymes, microbes, herbicides, zeolites, silicates, calcium carbonate, sodium carbonate, sodium bicarbonate, sodium sesquicarbonate, magnesium salts, silicates, aluminosilicates, other peroxides, and mixtures thereof,
said solid form preferably having a bulk density greater than 0.65 g/cm³, the ingredients of the combination product being present in admixture or separately in one or more containers.

24. The product according to claim 23 wherein the solid form sinks in water at temperatures of from 0° C. to 99° C.

25. The product according to claim 23, wherein the solid form is granules and flakes.

26. The product according to claim 23, wherein the inorganic peroxide is at least one peroxide selected from the group consisting of calcium peroxide, strontium peroxide, barium peroxide, zinc peroxide, cadmium peroxide, sodium peroxide, and magnesium peroxide.

27. The product according to claim 26, wherein the inorganic peroxide is a peroxide selected from the group consisting of calcium peroxide, magnesium peroxide, zinc peroxide, sodium peroxide, and mixtures thereof.

28. The process according to claim 1, wherein said solid form is combined with reactants used in a hydroxyl free radical generation reaction, the reactants being added before, with or after the solid form.

29. The process according to claim 1, wherein the solid form is used in combination with enzymes, microbes, or mixtures thereof.

30. The process according to claim 1, whereby a higher dose of said solid form is used to reach acceptable water or sediment quality, followed by lower doses to maintain water quality.

31. The process according to claim 1, whereby the solid form is used in conjunction with an algaecide, said algaecide being added before, with or after the solid form.

32. The process according to claim 1, wherein the solid form is used with another method to increase dissolved oxygen, or addition of a peroxide.

33. The process according to claim 1, whereby the solid form is used with a second oxidizer to effect the oxidation of toxic organic contaminants, the second oxidizer being selected from the group consisting of sodium percarbonate, hydrogen peroxide, sodium persulfate, potassium persulfate, and their mixtures, this second oxidizer being added before, with or after the solid form, optionally by using in addition a product to adjust the pH.

34. The process according to claim 33, whereby a metal compound is added before, with or after the other products.

35. A process for ex situ treatment of contaminated sediments and soils comprising:
adding at least one solid form comprising at least one inorganic peroxide and at least one corresponding hydroxide, oxide and/or carbonate, and optionally including one or more binders, extenders, adjuvants, buffers, or stabilizers, said solid form with a particle size at least 100 microns in average diameter.

36. The process according to claim 35 wherein the solid form has a mean particle size of at least 100 microns.
